(19) United States
(12) Patent Application Publication
(10) Pub. No.: US 2025/0134805 A1
(43) Pub. Date: May 1, 2025

(54) ENHANCED MUCOUS ADHESION VIA A COMPLEX OF CARBOXYMETHYL STARCH AND PULLULAN POLYMERS

(71) Applicant: Professional Compounding Centers of America, Inc., Houston, TX (US)

(72) Inventor: Daniel BANOV, Sugarland, TX (US)

(21) Appl. No.: 18/494,551

(22) Filed: Oct. 25, 2023

(51) Int. Cl. A61K 9/00; A61K 47/36

(52) U.S. Cl. CPC A61K 9/006; A61K 47/36

(57) ABSTRACT

Compositions and methods of using and manufacturing a pharmaceutical powder and a pharmaceutical hydrogel are provided. The pharmaceutical powder may form a gel after the addition of water. The gel may form a protective film upon drying that can coat the site of application. The protective film may reform a gel after the addition of water to facilitate cleaning and easy removal from the application surface. The pharmaceutical powder and pharmaceutical hydrogel may include pullulan (e.g., in an amount of from about 1 wt. % to about 30 wt. %) and sodium carboxymethyl starch (e.g., in an amount of from about 2 wt. % to about 50 wt. %). The pharmaceutical powder and pharmaceutical hydrogel improve mucoadhesion and wound adhesion and may be combined with active pharmaceutical ingredients (APIs) to treat mucous membrane or dermal diseases or wounds.

### FIELD OF THE INVENTION

The present disclosure relates to pharmaceutical compositions that improve the application and removal of active pharmaceutical ingredients (APIs) to the mucous membrane. Specifically, the pharmaceutical composition discussed herein includes polymers, such as sodium carboxymethyl starch and pullulan.

### BACKGROUND

The mucous membrane is a lining that covers the inside of cavities and organs that are exposed to particles from the outside, such as invasive pathogens. The mucous membrane also provides lubrication and it is involved in absorption within the gastrointestinal, respiratory, and urogenital tracts. Mucous membranes comprise a surface layer, called the epithelial layer that is composed of epithelial cells. Epithelial cells secrete mucin, a mucopolysaccharide, for protection of the body. These cells are stacked in columns forming several layers of cells. The epithelial layer attaches to connective tissue that sits deeper in the body and includes structural protein molecules, veins and nerves. The mucous membrane's deepest layer comprises smooth muscle which allows the mucous membrane to stay in flux, thus allowing it to stretch and contract.

Mucoadhesion occurs when two surfaces, one being a mucous membrane and the other typically being the surface of a drug delivery system, are held together by interfacial forces for a prolonged period of time. Improvements in mucoadhesion not only have the potential to localize drug delivery, but also coat and protect damaged tissues. Mucoadhesive interactions are accomplished by chemical bonds and other interactions at the molecular level, including hydrogen bonding, hydrophobic interactions, and others. But they are greatly dependent on the type of formulation used such as liquid (e.g., an eye drop), semi-solid (e.g., a vaginal gel) or solid formulations (e.g., a tablet or patch).

Improved mucoadhesion is relevant when treating different pathological conditions or wounds. When a wound occurs, there is an opening in the skin or mucous membrane which enables the access of invasive pathogens into the tissues and can result in wound infections. Mucoadhesive polymers or delivery systems can provide a prolonged coating that can protect the tissues from the outside elements. Conventional treatments can include topical formulations that can act locally and therefore require smaller doses. In addition, lower drug doses can reduce side effects and the risk of affecting healthy bacteria in the body.

### SUMMARY

Compositions and methods are provided for a pharmaceutical powder and a pharmaceutical hydrogel. The disclosed compositions and methods improve mucous adhesion, making it beneficial for medical and pharmaceutical applications where prolonged mucosal contact is essential, by improving the application and removal of the API. As discussed in more detailed herein, the disclosed compositions and methods significantly accelerate healing times. The disclosed compositions and methods provide improvements in drug release, increase stability, and enhance biocompatibility.

In one example, a pharmaceutical powder includes sodium carboxymethyl starch and pullulan. Additionally, this particular pharmaceutical powder may include ectoin natural, trehalose 100, or beta-cyclodextrin.

In another example, a pharmaceutical hydrogel includes sodium carboxymethyl starch and pullulan. Additionally, this particular pharmaceutical hydrogel may include ectoin natural, trehalose 100, or beta-cyclodextrin.

The disclosed pharmaceutical powder may form a gel after the addition of water. The gel can form a protective film upon drying that can coat the site of application. The protective film may reform a gel after the addition of water to facilitate cleaning and easy removal from the application surface.

The disclosed pharmaceutical hydrogel may form a protective film at the application surface after application. The protective film may be rehydrated into the gel after addition of water, to facilitate cleaning of the surface or wound.

In one example, the pharmaceutical powder comprises pullulan in an amount of from about 1% to about 30% pullulan by weight, with 15% being preferred; and from about 2% to about 50% sodium carboxymethyl starch by weight, with 40% being preferred.

In one example, the pharmaceutical composition hydrogel comprises pullulan in an amount of from about 1% to about 30% pullulan by weight, with 15% being preferred; and from about 2% to about 50% sodium carboxymethyl starch by weight, with 40% being preferred.

In one example, the pharmaceutical composition, powder, and hydrogel may be combined with suitable active pharmaceutical ingredients (APIs) and produce a mucoadhesive pharmaceutical composition, powder, or hydrogel. The mucoadhesive pharmaceutical composition, powder, or hydrogel may be used in treatment of mucous membrane diseases and wounds.

In another example, the pharmaceutical composition, powder, and hydrogel are suitable for mammalian wound care, including dermal wound care, and application on mammalian mucous membranes, such as, for example nasal, oral, rectal, vaginal mucous membranes, and the like, for wound treatment thereon.

This summary is intended to introduce a selection of concepts in a simplified form that is further described in the detailed description section of this disclosure. The summary is not intended to identify key or essential features of the claimed subject matter, nor is it intended to be an aid in determining the scope of the claimed subject matter. Additional objects, advantages, and novel features of the technology will be set forth in part in the description that follows, and in part will become apparent to those skilled in the art upon examination of the disclosure or learned through practice of the technology

### BRIEF DESCRIPTION OF THE DRAWINGS

Implementations of the present disclosure are described in detail below with reference to the attached drawing figures, wherein:

FIG. 1 depicts a graph illustrating percent cell viability vs exposure time on reconstructed human epidermis of two pharmaceutical compositions for comparison;

FIG. 2 depicts a graph illustrating the cumulative release of Metronidazole 2% vs time of two pharmaceutical compositions for comparison;

FIG. 3 depicts images 1-4 that illustrate keratinocyte migration (green fluorescence) for Phenytoin and Misoprostol in a control sample, and two pharmaceutical compositions for comparison;

FIG. 4 depicts images 1-9 of the wound areas for selected mice in a control group and test groups of treatment with two pharmaceutical compositions for comparison at day zero (before treatment) and days 3 and 9 of treatment;

FIG. 5 depicts a graph illustrating the mean percentage of relative wound areas for the mice in the control group and test groups of treatment with two pharmaceutical compositions for a total of 9 days;

FIG. 6 depicts images A-D of the right side of an ankle before treatment, 1 and 6 weeks post-treatment and at the end of treatment with a pharmaceutical powder composition; and

FIG. 7 depicts images 1-5 of a dermal wound treatment using a composition described herein.

### DETAILED DESCRIPTION

Glycoproteins are a major component of the mucous membrane, responsible for its gel structure, and can interact with mucoadhesive polymers. Common interactions between glycoproteins and mucoadhesive polymers include non-covalent interactions such as ionic interactions or chain entanglements.

Mucoadhesive polymers can form strong intermolecular hydrogen bonding with the mucous membrane and in some instances penetrate the mucus network. Mucoadhesive polymers can be classified into two categories: specific and non-specific polymers. Specific bioadhesive polymers include lectins and fimbrin polymers that can adhere to a specific chemical structure within the biological molecules. Non-specific bioadhesive polymers include chitosan, poly(acrylic acid) and polycyanoacrylate, and can adhere to both cell surfaces and the mucous layer.

Some examples of polymers are pullulan and sodium carboxymethyl starch. Both pullulan and sodium carboxymethyl starch have excellent physicochemical and mechanical properties. Sodium carboxymethyl starch is a biodegradable and water soluble polysaccharide. Pullulan is a polysaccharide comprising maltotriose units that are interlinked by α 1-6-glycosidic bonds. Pullulan is also highly water soluble in nature and has film-forming properties compared to other polysaccharides.

Some examples of pathological conditions that could benefit from a pharmaceutical compound that improves application and removal of APIs include herpes, hemorrhoids, chronic otitis, vaginal atrophy and others. For example, herpes simplex virus is a common infection that can cause ulcers and painful blisters. Most people have only mild symptoms but more severe symptoms can also be experienced such as fever, body aches and swollen lymph nodes. Treatment of herpes simplex virus varies, but can include antiviral medicines such as famciclovir and acyclovir. However, oral medications can sometimes have limited effectiveness because of poor solubility and systemic side effects. Topical medicines such as lidocaine and benzocaine or topical (gels or creams) analgesic such as a benzydamine mouthwash can be prescribed to reduce pain. Topical medicines may present improved efficacy if their permeation and adhesion time can be enhanced. Additionally, topical medicines may be advantageous if they facilitate application into the treatment area and cleaning of the surface.

The disclosed composition may be in the form of a powder. This is beneficial because an anhydrous product uses relatively less or does not use preservatives to protect the formula since it has low water activity, e.g., lower than 0.6, and also because preservatives can sometimes interact with active ingredients like antibiotics, affecting their efficacy. This is also beneficial because the powder can be reconstituted at the time of use to form a gel with minimal shear, making it extremely easy to apply this gel onto the wound. Upon application, the gel starts to form a protective film over the wound. While it forms a film post-application, it can easily revert to its gel state upon contact with water. This feature is particularly beneficial for wound cleaning.

In addition, this is beneficial because some APIs such as, for example, inulin, lidocaine, and benzocaine are present in powder form which may facilitate incorporation into the pharmaceutical formulation. In addition, this is beneficial because the powder composition may be applied directly to the mucous membrane that is being treated. In some aspects, the pharmaceutical powder may be easily combined with different APIs immediately prior to its application, which broadens the possibility of the different APIs that may be combined with the pharmaceutical powder. This pharmaceutical powder is also beneficial because, in this example, it is shelf stable and may easily be applied through various delivery mechanisms. The disclosed pharmaceutical powder may form a gel after the addition of water. In some cases, this is beneficial because water may be added directly into the powder in the mucous membrane to form the gel directly at the treatment location. The gel, when allowed to air dry, may form a protective film which is formed at the exact treatment location. The formation of the protective film is advantageous to provide immediate treatment and protection to the mucous membrane being treated. The protective film may reform a gel after addition of water, which is beneficial because it facilitates cleaning and easy removal of the pharmaceutical composition from the application site.

The disclosed composition may be in the form of a hydrogel. In some aspects, this is beneficial because the pharmaceutical hydrogel is ready to be applied onto the treatment location, skipping a gel formation step. The disclosed pharmaceutical hydrogel may form a protective film at the application surface after application, when allowed to air dry. The protective film provides a barrier to the mucous membrane from any invasive pathogens and prolonged contact of the therapeutic. The protective film may be rehydrated into the gel after addition of water, which is beneficial because it facilitates cleaning of the surface or wound.

Pullulan is an extracellular microbial polysaccharide consisting of maltotriose units, also known as α-1,4; α-1,6-glucan. Pullulan is often used for coating, glazing and as a film forming agent. Pullulan in pure form is colorless, odorless, tasteless and transparent. Pullulan is also highly water soluble, fat resistant and shows low oil and oxygen permeability. Excipient compositions including pullulan produce a strong addition to mucous membranes.

Sodium carboxymethyl starch (CMS) is a polysaccharide that is biodegradable and non-toxic. CMS is also water soluble at room temperature. CMS has been extensively studied for drug delivery, particularly for oral administration.

Trehalose is a disaccharide made out of two glucose molecules. Trehalose has high water retention capabilities which broadens its application to different industries. Trehalose is effective for protection of cells against desiccation. Damage and denaturalization can result from desiccation and oxidative stress of the cellular membranes and labile proteins, and trehalose has the ability to protect against it.

Hydrogels are a network of polymer chains with great water absorbance ability, allowing them to swell when placed on an aqueous medium and retain the absorbed volume of water. Hydrogels have potential in a wide array of applications because they can take different physical forms, such as solid molded forms, pressed powder matrices, different types of coatings and microparticles, or others.

Active pharmaceutical ingredients (APIs) are a component or a compound that induces a particular or intended effect in diagnosis, cure, mitigation, disease prevention or treatment, or has an effect in restoring, correcting, or modifying physiological functions of a human, animal, or other living creature.

In some examples of use, the pharmaceutical composition may be combined with APIs for treating particular conditions. Examples include but are not limited to analgesics, anesthetics anthelmintics, anti-fungals, antihistamines, anti-inflammatory agents, antimycobacterial agents, antineoplastic agents, antiviral agents, astringents, chemotherapy agents, corticosteroids, dermatological agents, hormones, muscle relaxants, or any other API, or mixtures of any two or more thereof.

As used herein, “wt. %” means the percent concentration of the component in the formulation measured on a weight-to-weight basis. For example, 1 wt. % of component A=[(mass of component A)/(total mass of the pharmaceutical composition including the mass of component A)]×100.

Reference to “about” a value or parameter herein refers to the usual error range for the respective value readily known to the skilled person in this technical field.

An example of the present disclosure may include a pharmaceutical powder. The pharmaceutical powder includes sodium carboxymethyl starch and pullulan. Additionally, this particular pharmaceutical powder may include various components, including, but not limited to, ectoin natural, trehalose 100, beta-cyclodextrin, or other components.

The pharmaceutical powder comprises pullulan in an amount of from about 1 wt. % to about 30 wt. % and sodium carboxymethyl starch in an amount of from about 2 wt. % to about 50 wt. %. In some aspects, pullulan may be present in an amount of from about 1 wt. % to about 25 wt. %, or from about 1 wt. % to about 20 wt. %, or from about 5 wt. % to about 25 wt. %, or from about 10 wt. % to about 30 wt. %, or from about 10 wt. % to about 20 wt. %, or from about 10 wt. % to about 15 wt. %, or from about 15 wt. % to about 20 wt. %. Sodium carboxymethyl starch may be present in an amount of from about 2 wt. % to about 45 wt. %, or from about 2 wt. % to about 40 wt. %, or from about 10 wt. % to about 35 wt. %, or from about 10 wt. % to about 30 wt. %, or from about 10 wt. % to about 25 wt. %, or from about 10 wt. % to about 20 wt. %, or from about 10 wt. 5 to about 15 wt. %, or from about 20 wt. % to about 40 wt. %, or from about 20 wt. % to about 35 wt. %, or from about 20 wt. % to about 30 wt. %, or from about 20 wt. % to about 25 wt. %, or from about 15 wt. % to about 20 wt. %, or from about 30 wt. % to about 40 wt. %, or from about 30 wt. % to about 45 wt. %.

An example of the present disclosure may include the pharmaceutical powder comprising pullulan in an amount of from about 5 wt. % to about 30 wt. %, sodium carboxymethyl starch in an amount of from about 10 wt. % to about 50 wt. %, and trehalose in an amount of from about 0.5 wt. % to about 20 wt. %.

In a different example, the pharmaceutical powder comprises pullulan in an amount of from about 5 wt. % to about 20 wt. % and sodium carboxymethyl starch in an amount of from about 30 wt. % to about 45 wt. %.

In another example, the pharmaceutical powder comprises a preferred amount of pullulan of about 15 wt. % and sodium carboxymethyl starch of about 40 wt. %.

In one example, the pharmaceutical powder may be combined with suitable active pharmaceutical ingredients (APIs) and produce a mucoadhesive pharmaceutical composition powder. The mucoadhesive pharmaceutical composition powder may be used in treatment of mucous membrane diseases and wounds.

In another example, the pharmaceutical powder is suitable for application on mammalian mucous membranes, such as, for example nasal, oral, rectal, vaginal mucous membranes, and the like.

An example of the present disclosure includes a pharmaceutical hydrogel. The pharmaceutical hydrogel includes sodium carboxymethyl starch and pullulan. Additionally, this particular pharmaceutical hydrogel may include various compounds, including, but not limited to, ectoin natural, trehalose 100, beta-cyclodextrin or other compounds.

The pharmaceutical hydrogel comprises pullulan in an amount of from about 1 wt. % to about 30 wt. % and sodium carboxymethyl starch in an amount of from about 2 wt. % to about 50 wt. %. In some aspects, pullulan may be present in an amount of from about 1 wt. % to about 25 wt. %, or from about 1 wt. % to about 20 wt. %, or from about 5 wt. % to about 25 wt. %, or from about 10 wt. % to about 30 wt. %, or from about 10 wt. % to about 20 wt. %, or from about 10 wt. % to about 15 wt. %, or from about 15 wt. % to about 20 wt. %. Sodium carboxymethyl starch may be present in an amount of from about 2 wt. % to about 45 wt. %, or from about 2 wt. % to about 40 wt. %, or from about 2 wt. % to about 35 wt. %, or from about 2 wt. % to about 30 wt. %, or from about 2 wt. % to about 25 wt. %, or from about 10 wt. % to about 20 wt. %, or from about 10 wt. 5 to about 15 wt. %, or from about 20 wt. % to about 40 wt. %, or from about 20 wt. % to about 35 wt. %, or from about 20 wt. % to about 30 wt. %, or from about 20 wt. % to about 25 wt. %, or from about 15 wt. % to about 20 wt. %, or from about 30 wt. % to about 40 wt. %, or from about 30 wt. % to about 45 wt. %.

An example of the present disclosure may include the pharmaceutical hydrogel comprising pullulan in an amount of from about 5 wt. % to about 30 wt. %, sodium carboxymethyl starch in an amount of from about 10 wt. % to about 50 wt. %, and trehalose in an amount of from about 0.5 wt. % to about 20 wt. %.

In a different example, the pharmaceutical hydrogel comprises pullulan in an amount of from about 5 wt. % to about 20 wt. % and sodium carboxymethyl starch in an amount of from about 30 wt. % to about 45 wt. %.

In another example, the pharmaceutical hydrogel comprises a preferred amount of pullulan of about 15 wt. % and sodium carboxymethyl starch of about 40 wt. %.

In one example, the pharmaceutical hydrogel may be combined with suitable active pharmaceutical ingredients (APIs) and produce a mucoadhesive pharmaceutical composition hydrogel. The mucoadhesive pharmaceutical hydrogel may be used in treatment of mucous membrane diseases, and wounds.

In another example, the pharmaceutical hydrogel is suitable for application on mammalian mucous membranes, such as, for example nasal, oral, rectal, vaginal mucous membranes, and the like.

An example of the method of using a pharmaceutical powder is presented. The method of using may include applying a pharmaceutical powder onto a mucous membrane or dermal surface. The pharmaceutical powder may be pre-mixed with an API prior to application into the mucous membrane or dermal surface. The pharmaceutical powder may form a gel after addition of water. Water may be sprayed directly into the powder at the mucous membrane or dermal surface. The gel may be formed directly at the mucous membrane or dermal surface, or reconstituted prior to application onto the mucous membrane or dermal surface. In some examples, bandages may be applied over the gel to cover the mucous membrane or dermal surface, or in some examples the gel may be allowed to air dry without any bandages. The gel, when applied into the mucous membrane or dermal surface, may form a protective film after drying. The protective film may be rehydrated into the gel after addition of water. The reversible behavior facilitates cleaning and removal of the pharmaceutical powder from the mucous membrane or dermal surface.

In another example of the method of using, a pharmaceutical hydrogel is applied to a mucous membrane or dermal surface. The method includes applying the pharmaceutical hydrogel onto a mucous membrane or dermal surface in need of treatment. The pharmaceutical hydrogel may be combined with one or more APIs previous to application or during the application step. The pharmaceutical hydrogel in the mucous membrane may dry and form a protective film. The protective film creates a barrier for isolating the mucous membrane form invasive pathogens and provides a uniform surface for prolonged contact with the therapeutic. The protective film may reform the hydrogel after addition of water, which facilitates cleaning and removal.

The embodiments disclosed herein, including the pharmaceutical powder, pharmaceutical hydrogel and methods of using corresponding to the pharmaceutical compositions, may be further understood by reference to the following non-limiting examples.

### EXAMPLES

Example 1 illustrated below is one example of a pharmaceutical powder that exhibits particular mucoadhesive properties. The table below includes components and an amount (wt. %) of each component.

TABLE 1

| Component | wt. % |
|---|---|
| PULLULAN | 15.0% |
| SODIUM CARBOXYMETHYL STARCH | 40.0% |
| TREHALOSE 100 | 15.0% |
| ECTOIN NATURAL | 7.0% |
| BETA-CYCLODEXTRIN | 13.0% |
| CBP INULIN HD | 10.0% |
| Totals | 100.00% |

Example 2 illustrated below describes an evaluation of the safety and toxicological profile on reconstructed human epidermis. The epidermis is the outermost skin layer and it is increasingly used as a route of drug administration. Topical compounded medications must be non-toxic and non-irritant to the skin and, therefore, it is important to guarantee the safety of the bases used in compounding. The aim of this example is to evaluate the safety and toxicological profile of the pharmaceutical hydrogel comprising 3 wt. % pullulan and 8 wt. % sodium carboxymethyl starch, in comparison to a commercially available drug, a surfactant having a composition of poly(ethylene oxide) and poly(propylene oxide) widely used in wound care, using a 3-dimensional (3D) in vitro model of reconstructed human epidermis: EpiDerm™ by MatTek Corporation (Ashland, MA), a highly differentiated 3D model which consists of human-derived epidermal keratinocytes, cultured and differentiated to resemble the human epidermis.

Upon receipt of the MTT-100 kit R, the EPI-200 cells (Lot 39169) were maintained in the supplied culture media and stored in accordance to the manufacturer's protocol until the initiation of the study. Following preparation of the cells, the EpiDerm™ tissues were treated in triplicate with 100 μL of the test the pharmaceutical hydrogel 20% and another set of tissues were treated with the commercially available drug 20% for 4, 16 and 24 hours. A triplicate set of EpiDerm™ tissues was also left untreated to serve as negative control. Following the exposure period, the dosing solutions were removed and the cells were analyzed for cell viability by the MTT Effective Time 50 (ET₅₀) assay, which consists of measuring the reduction of MTT (3-[4,5-dimethylthiazol-2yl]-2,5-diphenyltetrazolium bromide) by the cells. Succinate dehydrogenase enzymes within the mitochondria of viable cells have the ability to reduce soluble yellow tetrazonium salt of MTT to an insoluble purple formazan derivative. MTT is therefore an indicator of cell viability as the tissues are evaluated for their ability to reduce soluble-MTT (yellow) to formazan-MTT (purple).

Viability of the epidermis tissue cells following exposure to the test products is represented by the absorbance of the respective extracts and expressed in percentage relative to the negative control, as follows: % Cell Viability=100×[OD (test product)/OD (negative control)]. The greater the absorbency of the extracts, the greater the amount of MTT reduced by succinate dehydrogenase and, as a result, the higher the percent cell viability within the tissue.

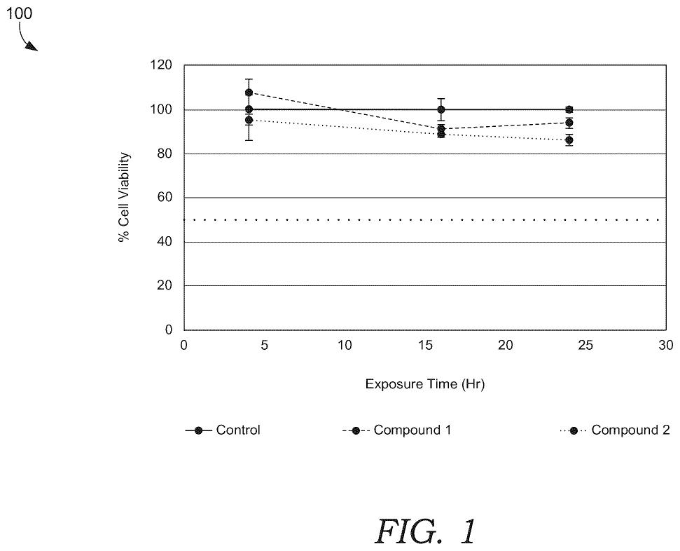

Upon 24 hours of study, the viability of the cells exposed to the pharmaceutical hydrogel was superior to 90%. Similarly, the viability of the cells exposed to the commercially available drug, a surfactant widely used in wound care, was superior to 85%, as demonstrated in FIG. 1.

The toxic exposure time (ET₅₀) is the time when cell viability is reduced to 50%, which is represented by α dashed line in the FIG. 1. The general guideline for correlation of in vitro and in vivo results states that products with an ET₅₀ of 24 hours are expected to be non-irritant. According to the results obtained, the ET₅₀ of both the pharmaceutical hydrogel and the commercially available drug is superior to 24 hours and, therefore, both products have a good safety and toxicological profile.

In conclusion, the pharmaceutical hydrogel does not cause toxicity to the epidermis tissue. As a result, compounded medicines including this pharmaceutical hydrogel may be applied to the skin without causing any toxicity to the epidermis tissue.

Example 3 illustrates the in vitro drug release of Metronidazole 2% from the pharmaceutical hydrogel, comprising 3 wt. % pullulan and 8 wt. % sodium carboxymethyl starch, in comparison to a readily available drug in gel form. The in vitro drug release is a performance test for topical drug products used to measure the release rate of active pharmaceutical ingredients (APIs) from semisolid dosage forms. It is important to test the in vitro drug release of the pharmaceutical hydrogel to ensure its performance and comparability to a product of reference. This test is not intended though to predict in vivo performance, as opposed to the skin percutaneous absorption studies, since the primary factor that impacts bioavailability and clinical performance is skin permeation. However, this test can detect in vitro changes, as a result of formulation differences, that may correspond to altered in vivo performance of the dosage form. For this reason, its main purpose and use is comparison testing in which any difference in delivery rate is undesirable. This test is required by the FDA to determine the acceptability of minor processes and/or formulation changes in commercially approved semisolid dosage forms. The Unites States Pharmacopoeia (USP) recognizes different apparatus for the in vitro drug release test in the monograph <1724> Semisolid Drug Products-Performance Tests. The aim of this study is to evaluate and compare the in vitro drug release of Metronidazole 2% topical pharmaceutical hydrogel and Metronidazole 2% in a readily available gel that is a well-established and referenced base used in wound management therapy.

The in vitro drug release test was performed using the Franz Diffusion System (PermeGear, Inc.) using vertical diffusion cells (VDC) including 6-cell units. Each VDC cell assembly consisted of two chambers (donor and receptor chambers) separated by α membrane and held together by α clamp. The test samples (600 mg) sat on a synthetic, inert, highly permeable support membrane, intended to keep the test samples and receptor medium separate. A heating water circulator was used to maintain the temperature controlled at 37° C.±1.00° C. The 6-cell units operated together at one time (i.e., single run). The receptor medium samples were collected at 0.5, 1, 2, 3, 4, 5 and 6 hours (hr) by stopping the stirrer, withdrawing 1 mL of sample, and replacing the same volume with stock receptor medium

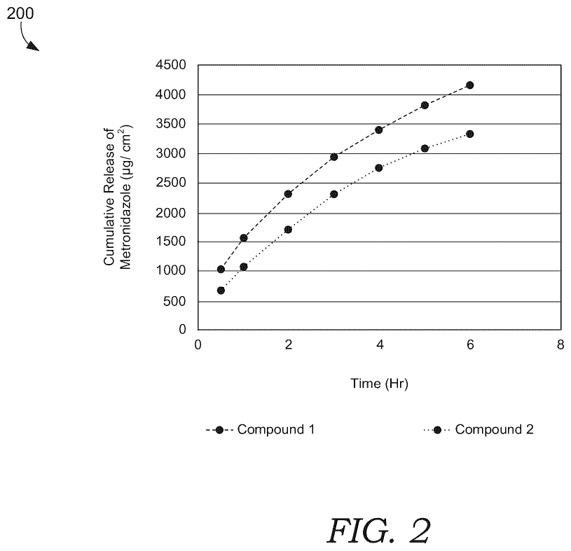

For each cell unit, the amount of metronidazole released (μg/cm²) was determined at each sampling time, and the cumulative amount of metronidazole released was plotted versus time (hr). Metronidazole 2% exhibited a similar in vitro release profile from both the pharmaceutical hydrogel and the readily available gel throughout the study period of 6 hours. The amount released from the pharmaceutical hydrogel was slightly higher at all time points in comparison to the readily available gel. By the end of the study, a total of 7,350.70±49.88 μg/cm² (61.26%) and 5,905.56±457.17 μg/cm² (49.21%) of metronidazole had been released from the pharmaceutical hydrogel and the readily available gel, respectively as seen from FIG. 2.

This comparative example was not designed to evaluate any statistical differences between the two bases. Instead, it is able to provide qualitative insights on the drug release performance of the bases. In conclusion, the in vitro drug release test has demonstrated that metronidazole 2% has comparable release profiles when incorporated in the well-established readily available gel versus the pharmaceutical hydrogel.

Example 4 describes and in vitro evaluation of wound healing by Phenytoin 2% and Misoprostol 0.0024% in a pharmaceutical hydrogel, comprising 3 wt. % pullulan and 8 wt. % sodium carboxymethyl starch, in comparison to a readily available gel. Re-epithelialization is a process in wound healing that involves the migration of keratinocytes (cells within the epidermal layer of the skin) from the edge, towards the centre of the wound, to form a thin layer of cells over the exposed area. The rate at which keratinocyte migration occurs is important in wound healing as it is the body's first attempt at restoring the protective skin layer. Delays in this healing process may result in wound infections and hypertrophic skin scarring. The purpose of this example is to assess the ability of the test formulations Phenytoin 2% and Misoprostol 0.0024% in the pharmaceutical hydrogel, and Phenytoin 2% and Misoprostol 0.0024% in a readily available gel, to facilitate keratinocyte migration by evaluating in vitro the process of re-epithelialization, using primary human keratinocytes.

The in vitro evaluation was performed using the Oris™ cell migration assay kit (Platypus Technologies, Inc.), which consists of a 96-well plate, cell seeding stoppers that inhibit the spread of cells into the migration zone (center of the wells), and a black mask that allows for detection of cell migration. The cells were treated with the test formulations for 24 hours and then stained with Calcein AM, a non-fluorescent dye that is converted to green fluorescent calcein by viable cells. Fluorescence was detected using the CLARIOstar® plate reader (BMG Labtech) with Stars software for analysis at 483/14 excitation and 530/30 emission wavelength.

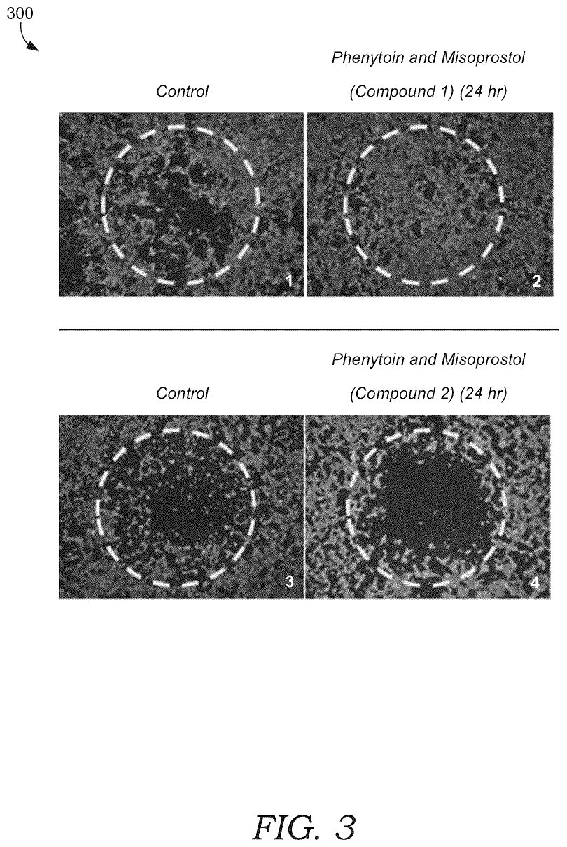

The abilities of the test formulations to facilitate migration of primary human keratinocytes into the migration zone was quantified based on the green fluorescence detected by the plate reader, which is expressed in terms of Relative Fluorescence Unit (RFU). Phenytoin 2% and Misoprostol 0.0024% in the pharmaceutical hydrogel at 24 hours showed a mean change of 70.62% from control, which means that the topical hydrogel significantly promoted cell migration. In the contrary, Phenytoin and Misoprostol in the readily available gel did not increase cell migration (negative mean change) as shown in FIG. 3. Keratinocyte migration is part of the re-epithelialization process in wound healing and, therefore, the pharmaceutical hydrogel formula is likely to have greater wound healing abilities than the corresponding formula for the readily available gel.

Example 5 illustrates an in vivo evaluations of wound healing by Phenytoin 2% and Misoprostol 0.0024% in a pharmaceutical hydrogel, comprising 3 wt. % pullulan and 8 wt. % sodium carboxymethyl starch, in comparison to a readily available gel. Chronic wounds are a major clinical problem that leads to considerable morbidity and mortality worldwide. Newly developed products are subjected to both in vitro and in vivo studies to ensure its safety and efficacy in wound management. A commonly used in vivo test is the diabetic mice wound healing model (BKS-db), including both control group and test group(s).

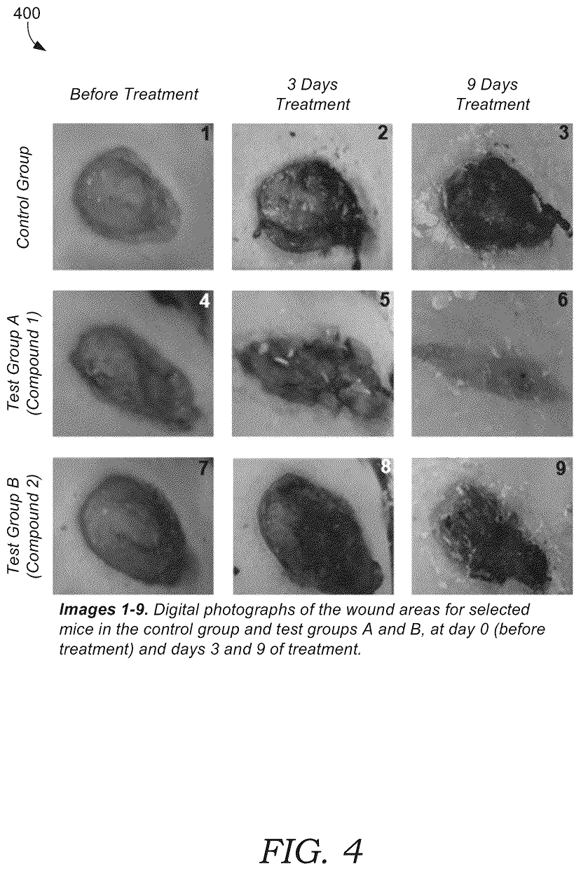

The in vivo evaluation test was conducted by GemPharmatech Co., Ltd following ethics approval: certification number 511214900020707; animal protocol GPTAP20230810-4; project number PO-GJC052023078305-01. The test products Phenytoin 2% and Misoprostol 0.0024% Topical pharmaceutical hydrogel (A), and Phenytoin 2% and Misoprostol 0.0024% in a readily available Gel (B) were provided. C57BL/6J/BKS-db male diabetic mice (n=12) were divided in 3 groups (n=4) according to blood glucose and body weight: control group, test group A and test group B. All mice were anesthetized and the hair on their dorsal skin was shaved. Two full-thickness excisional skin wounds were made to the back of each mouse using an 8-mm biopsy punch. Subsequently, 300 mg of the test product A and B were applied daily on the skin wounds of the mice in the test groups A and B, respectively, for a total of 9 days. The images on FIG. 4 show the skin wounds on days 0, 3 and 9. The wound areas were measured, normalized as percentage of day 0 and expressed as mean±standard deviation (mean±SD). The statistical analysis was performed using a T-test in which p<0.05 is considered statistically significant.

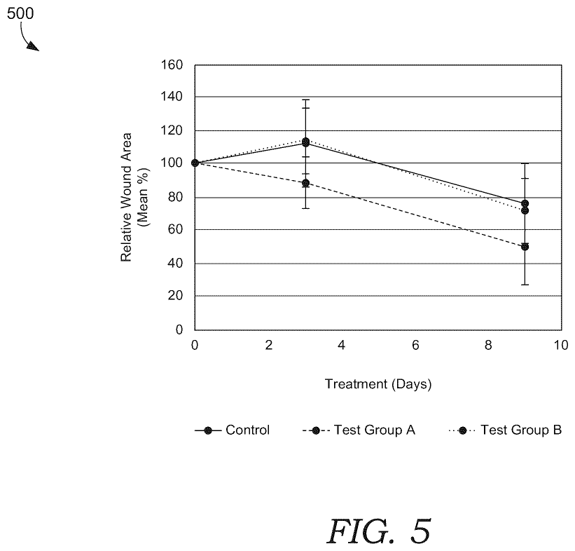

The mice were successfully treated for 9 days in the test groups A and B, as shown in FIGS. 4-6 and 7-9, respectively. The mice in the control group (Images 1-3 in FIG. 4) developed a slower wound healing response, in comparison to the treated mice. The test group A [Phenytoin and Misoprostol Topical Pharmaceutical Hydrogel]-demonstrated a more effective treatment response in comparison to the test group B (Phenytoin and Misoprostol in a readily available Gel). FIG. 5 shows a lower mean percentage of relative wound area at day 9 for the test group A (49.67%), when compared to the test group B (71.54%) and the control group (75.44%). These mean percentage results obtained are consistent with Images 1-9 in FIG. 4.

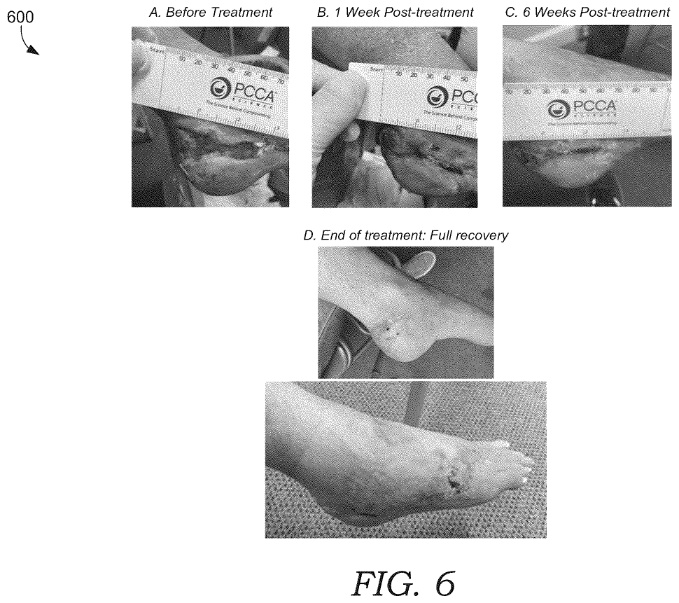

Example 6 illustrates the management of foot chronic wounds with the topical pharmaceutical powder, comprising 14.0 wt. % pullulan and 37.6 wt. % sodium carboxymethyl starch. A 53-year-old healthy female suffered an accident while standing next to her car to let the dog out. The car rolled over her right foot causing extensive wounds and rupture of her achilles tendon. Following two surgeries, the chronic wounds located on the back of the heel (achilles tendon), ankle (right side), and sole were still extensive and infected. The largest wound was the ankle with 3 inches by 1½ inches wide (Image A FIG. 6). The wound on the back of the heel measured 1½ inch. When the local compounding pharmacist was consulted, an innovative topical treatment was recommended, including pentoxifylline, phenytoin, naltrexone HCl, misoprostol, arginine HCl and beta glucan in the topical pharmaceutical powder (Table 2). FIG. 6 contains images that show the progression of the wound healing with the topical pharmaceutical powder as treatment.

TABLE 2

| Component | Amount | |
|---|---|---|
| Pentoxifylline USP | 2 | g |
| Phenytoin USP | 2 | g |
| Naltrexone Hydrochloride USP Anhydrous | Calculate | |
| Misoprostol 1% (HPMC Dispersion) | 0.24 | g |
| Arginine Hydrochloride USP | 1 | g |
| Beta Glucan (1,3) NQ | 0.2 | g |

| | |
|---|---|
| Base, Pharmaceutical Powder | q.s. 100 g |

The subject matter of the present invention is described with specificity herein to meet statutory requirements. However, the description itself is not intended to limit the scope of this disclosure. Rather, the inventors have contemplated that the claimed or disclosed subject matter might also be embodied in other ways, to include different steps or combinations of steps similar to the ones described in this document, in conjunction with other present or future technologies.

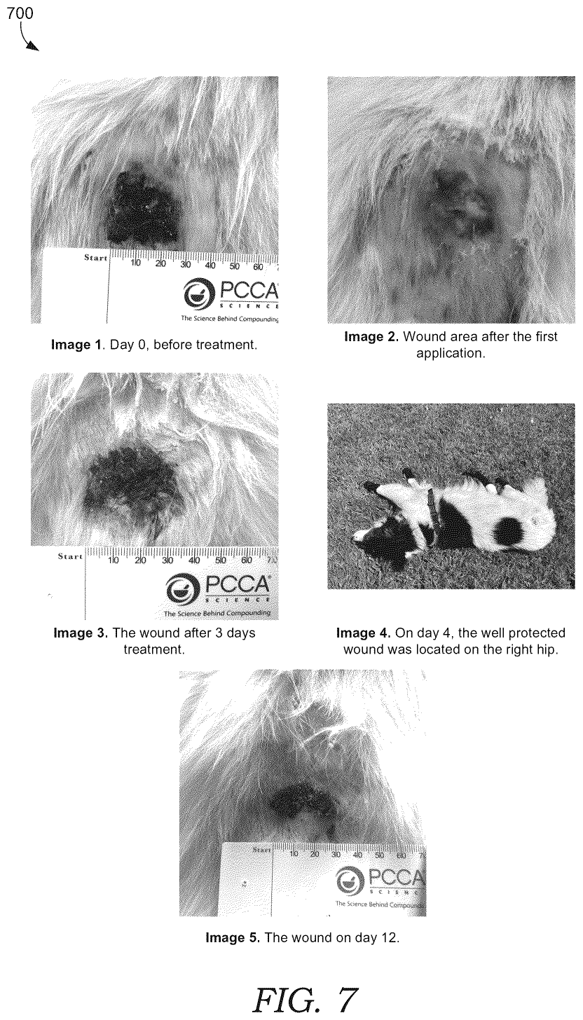

Example 7 illustrates treatment of a decubitus ulcer in a 15-year old border collie. The decubitus ulcer (pressure sore) on the bony part of the dog's right hip developed due to limited mobility after a stroke. The wound started from inflamed skin, quickly grew larger, and became an open wound with bleeding and necrotic skin tissue (FIG. 7, Image 1). The dog takes prednisone daily for Addison's disease, which also contributed to delayed healing of the wound.

Four days after the ulcer started, a formula containing 2% mupirocin, 0.1% tetracaine, and 1% tranexamic acid was prepared with the pharmaceutical powder, comprising 14.0 wt. % pullulan and 37.6 wt. % sodium carboxymethyl starch, and was given to the dog's owner in a collapsible bottle. The owner was instructed to clean the wound first, then cover it with a thin layer of the powder formula, spray a small amount of water to form a gel on top of the wound, and repeat until the entire wound is well covered. The picture after the first application is shown in FIG. 7 (Image 2). No bandage was needed as a firm protective layer was formed on top of the wound by the formula.

The wound area was flushed with water, cleaned up, and reapplied with the formula every 3 days. The wound at day 4 is shown in FIG. 7 (Image 3). The wound has completely closed, and a firm scab has formed. The inflammation of the surrounding skin has disappeared. A whole-body picture is also included to show the well protected wound (FIG. 7, Image 4).

By day 12, only a small piece of scab was loosely attached to the skin. New skin and hair have been growing in the healed area (FIG. 7, Image 5). No adverse reactions were observed during the treatment.

For purposes of this disclosure, the word “including,” “having,” and other like words and their derivatives have the same broad meaning as the word “comprising.” In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both (a or b thus includes either a or b, as well as a and b).

Some example aspects of the invention that may be practiced from the forgoing disclosure include the following:

Aspect 1: A pharmaceutical powder or hydrogel comprising: from about 1 wt. % to about 30 wt. % pullulan; and from about 2 wt. % to about 50 wt. % sodium carboxymethyl starch.

Aspect 2: Aspect 1, further comprising: from about 5 wt. % to about 30 wt. % pullulan; and from about 10 wt. % to about 50 wt. % sodium carboxymethyl starch.

Aspect 3: Aspect 2, further comprising: from about 5 wt. % to about 20 wt. % pullulan; and from about 30 wt. % to about 45 wt. % sodium carboxymethyl starch.

Aspect 4: Aspect 3, further comprising: about 15 wt. % pullulan; and about 40 wt. % sodium carboxymethyl starch.

Aspect 5: Any of Aspects 1-4, further comprising trehalose.

Aspect 6: Aspect 5, further comprising from about 0.5 wt. % to about 20 wt. % trehalose.

Aspect 7: Any of Aspects 1-6, wherein the powder forms a gel after addition of water, and thereafter, forms a film upon drying of the water.

Aspect 8: Aspect 7, wherein the film rehydrates to the gel after addition of water to the film.

Aspect 9: Any of Aspects 1-6, wherein the hydrogel forms a film post-application.

Aspect 10: Aspect 9, wherein the film rehydrates to the hydrogel after addition of water to the film.

Aspect 11: A method of using a pharmaceutical powder, comprising: applying the pharmaceutical powder onto a mucosal membrane, wherein the pharmaceutical powder comprise; from about 1 wt. % to about 30 wt. % pullulan; and from about 2 wt. % to about 50 wt. % sodium carboxymethyl starch; and adding water to the pharmaceutical powder after application to form a gel, wherein the gel forms a film on the mucosal membrane after drying.

Aspect 12: Aspect 11, further comprising adding water to the film to rehydrate the film into the gel.

Aspect 13, Any of Aspects 11-12, wherein the pharmaceutical powder comprises: from about 5 wt. % to about 30 wt. % pullulan; and from about 10 wt. % to about 45 wt. % sodium carboxymethyl starch.

Aspect 14, Aspect 13, wherein the pharmaceutical powder comprises: from about 5 wt. % to about 20 wt. % pullulan; and from about 30 wt. % to about 45 wt. % sodium carboxymethyl starch.

Aspect 15, Aspect 13, wherein the pharmaceutical powder comprises: about 15 wt. % pullulan; and about 40 wt. % sodium carboxymethyl starch.

What is claimed is:

1. A pharmaceutical powder comprising:
from about 5 wt. % to about 30 wt. % pullulan; and
from about 10 wt. % to about 50 wt. % sodium carboxymethyl starch.

2. The pharmaceutical powder of claim 1, comprising:
from about 5 wt. % to about 20 wt. % pullulan; and
from about 30 wt. % to about 45 wt. % sodium carboxymethyl starch.

3. The pharmaceutical powder of claim 1, comprising:
about 15 wt. % pullulan; and
about 40 wt. % sodium carboxymethyl starch.

4. The pharmaceutical powder of claim 1, further comprising trehalose.

5. The pharmaceutical powder of claim 4, comprising from about 0.5 wt. % to about 20 wt. % trehalose.

6. The pharmaceutical powder of claim 4, comprising about 15 wt. % pullulan, about 40 wt. % sodium carboxymethyl starch, and about 15 wt. % trehalose.

7. The pharmaceutical powder of claim 1, wherein the powder forms a gel after addition of water, and thereafter, forms a film upon drying of the water.

8. The pharmaceutical powder of claim 7, wherein the film rehydrates to the gel after addition of water to the film.

9. A pharmaceutical hydrogel comprising:
from about 1 wt. % to about 30 wt. % pullulan; and
from about 2 wt. % to about 50 wt. % sodium carboxymethyl starch.

10. The pharmaceutical powder of claim 9, comprising:
from about 5 wt. % to about 20 wt. % pullulan; and
from about 30 wt. % to about 45 wt. % sodium carboxymethyl starch.

11. The pharmaceutical powder of claim 9, comprising:
about 15 wt. % pullulan; and
about 40 wt. % sodium carboxymethyl starch.

12. The pharmaceutical hydrogel of claim 9, further comprising trehalose.

13. The pharmaceutical hydrogel of claim 9, comprising from about 0.5 wt. % to about 20 wt. % trehalose.

14. The pharmaceutical hydrogel of claim 9, comprising about 15 wt. % pullulan, about 40 wt. % sodium carboxymethyl starch, and about 15 wt. % trehalose.

15. The pharmaceutical hydrogel of claim 9, wherein the hydrogel forms a film post-application.

16. The pharmaceutical hydrogel of claim 15, wherein the film rehydrates to the hydrogel after addition of water to the film.

17. A method of using a pharmaceutical powder, comprising:
applying the pharmaceutical powder onto a mucosal membrane or dermal surface, wherein the pharmaceutical powder comprise;
from about 5 wt. % to about 30 wt. % pullulan; and
from about 10 wt. % to about 50 wt. % sodium carboxymethyl starch; and
adding water to the pharmaceutical powder after application to form a gel, wherein the gel forms a film on the mucosal membrane or dermal surface after drying.

18. The method of claim 17, further comprising adding water to the film to rehydrate the film into the gel.

19. The method of claim 17, wherein the pharmaceutical powder comprises:
from about 5 wt. % to about 20 wt. % pullulan; and
from about 30 wt. % to about 45 wt. % sodium carboxymethyl starch.

20. The method of claim 17, wherein the pharmaceutical powder comprises:
about 15 wt. % pullulan; and
about 40 wt. % sodium carboxymethyl starch.
